(19) United States
(12) Plant Patent Application Publication
(10) Pub. No.: US 2012/0102612 P1
(43) Pub. Date: Apr. 26, 2012

(54) Gasteria plant named 'WT10'

(50) ;

(76) Inventor: Wander Tuinier, Krimpen a/d IJssel (NL)

(21) Appl. No.: 12/925,502

(22) Filed: Oct. 21, 2010

(51) Int. Cl. A01H 5/00

(52) U.S. Cl. PLT/373

(57) ABSTRACT

A new and distinct Gasteria hybrid cultivar named ‘WT10’ is disclosed, characterized by foliage with a strong green color and grey green spots. The overall plant form is a very evenly growing whorled rosette. The new variety is a Gasteria hybrid, typically produced as an ornamental plant.

### LATIN NAME OF THE GENUS AND SPECIES

Gasteria hybrid

### VARIETY DENOMINATION

‘WT10’

### BACKGROUND OF THE INVENTION

The new cultivar was discovered as a seedling, resulting from the 2007 crossing of a group of unnamed Gasteria hybrid as the seed parent and an unnamed species of Gasteria hybrid as the pollen parent ‘WT10’ was selected by the inventor, Wander Durk Tuinier, a citizen of the Netherlands, in a group of seedlings resulting from the aforementioned crossing. The new variety was discovered April 2008, in a commercial greenhouse in Swellendam, South Africa.

Asexual reproduction of the new cultivar ‘WT10’ was first performed in South Africa, at a commercial laboratory by tissue culture in September 2008. ‘WT10’ has since produced many generations and has shown that the unique features of this cultivar are stable and reproduced true to type.

### SUMMARY OF THE INVENTION

The cultivar ‘WT10’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘WT10.’ These characteristics in combination distinguish ‘WT10’ as a new and distinct Gasteria cultivar:

1. Strong green foliage color with small grey-green dots.

2. Distinctive evenly whorled rosetting plant form.

### COMMERCIAL COMPARISON

Plants of ‘WT10’ can be compared to the unpatented commercial variety, Gasteria acinacifolia. Plants are similar in most horticultural characteristics however ‘WT10’ has a more acute leaf apex than Gasteria acinacifolia. ‘WT10’ grows more rapidly and produces side shoots more easily. Additionally, the leaves of ‘WT10’ are softer than those of Gasteria acinacifolia.

Plants of ‘WT10’ can be compared to the unpatented commercial variety, Gasteria batesiana. Plants are similar in more horticultural characteristics however ‘WT10’ produces side shoots more easily. Additionally the overall plant habit of ‘WT10’ is evenly, whorled rosetting while Gasteria batesiana produces irregular rosettes.

### BRIEF DESCRIPTION OF THE PHOTOGRAPH

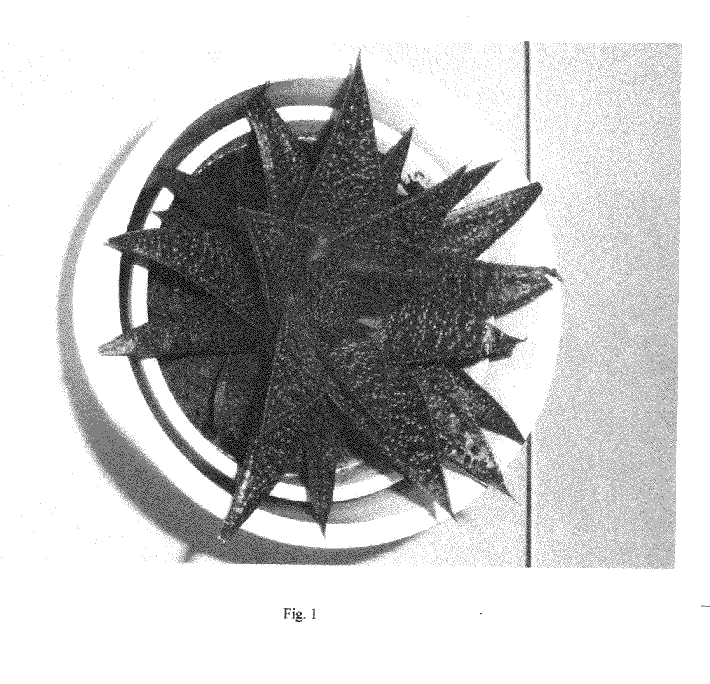

The accompanying photograph in FIG. 1 illustrates in full color a typical plant of ‘WT10’ indoors in the Netherlands. This plant is approximately 30 months old, shown in a 12.5 cm pot. The photograph was taken using conventional techniques and although colors may appear different from actual colors due to light reflectance it is as accurate as possible by conventional photographic techniques.

### DETAILED BOTANICAL DESCRIPTION

In the following description, color references are made to The Royal Horticultural Colour Chart, except where general terms of ordinary dictionary significance are used. The following observations and measurements describe ‘WT10’ plants in a commercial greenhouse in Kimpen aan de Ijssel, the Netherlands. Temperatures ranged from 20° C. to 22° C. night and day. No artificial light, photoperiodic treatments or chemical treatments were given to the plants. Measurements and numerical values represent averages of typical plant types.
Botanical classification: Gasteria hybrid ‘WT10’
Propagation:
   Time to initiate roots.—About 21 days at approximately 22-24° C.
   Root description.—Fibrous.
   Propagation method.—tissue culture.
Plant:
   Growth habit.—Rosulate.
   Age of plant described.—Approximately 30 months.
   Container size.—12.5 cm diameter.
   Height.—Approximately 7.7 cm to top of highest leaf, measured from the soil line.
   Plant spread.—Approximately 15.5 cm.
   Growth rate.—Slow.
   Branching characteristics.—No lateral branches, whole plant consists of a single rosette; new rosettes (average: 2) grow from the base.
Foliage:
   Leaf.—Arrangement: Rosette Average Length: Approximately 7.0 cm. Average Width: Approximately 2.7 cm. Shape of blade: Lanceolate Apex: Apiculate with a short aristate tip Base: Broad cuneate Margin: Denticulate Texture of top surface: Pustulate Texture of bottom surface: Pustulate Quantity of leaves per plant: Approximately 35 Color: Young foliage upper side: Near RHS Green N137A pustules near RHS Greyed-Green 192D, base of leaf near attachment point near RHS 161B Young foliage under side: Near RHS Green 139A, pustules near RHS Greyed-Green 192D, base of leaf near attachment point near RHS 161B Mature foliage upper side: Near RHS Green 139A, pustules near RHS Grey-Green 192D, base near point of attachment near RHS White N155C Mature foliage under side: Near RHS Green 139A pustules near RHS Greyed-Green 192D, base near point of attachment near RHS N155C Venation: Type: Linear. Venation color upper side: Near RHS Green 139A, base venation RHS White N155C Venation color under side: Near RHS Green 139A, base venation RHS White N155C.
Flower: No flowers observed to date.
Other characteristics:
   Seeds and fruits.—Not observed to date.
   Disease/pest resistance.—Neither resistance nor susceptibility to the normal pathogens of Gasteria has been observed.
   Temperature tolerance.—Tolerates temperatures from approximately 3° C. to at least 40° C.
   Drought tolerance.—Drought tolerant succulent.

What is claimed is:

1. A new and distinct cultivar of Gasteria hybrid plant named ‘WT10’ as herein illustrated and described.
